(19) United States
(12) Patent Application Publication
(10) Pub. No.: US 2016/0161581 A1
(43) Pub. Date: Jun. 9, 2016

(54) MAGNETIC RESONANCE IMAGING APPARATUS AND METHOD OF OPERATING SAME

(71) Applicant: Samsung Electronics Co., Ltd., Gyeonggi-do (KR)

(72) Inventors: Keum-yong OH, Gyeonggi-do (KR); Ki-wan KIM, Gyeonggi-do (KR); Young-ha KIM, Seoul (KR); Sung-pil JUNG, Gyeonggi-do (KR); Yang-lim CHOI, Gyeonggi-do (KR)

(21) Appl. No.: 14/955,274

(22) Filed: Dec. 1, 2015

(30) Foreign Application Priority Data
Dec. 4, 2014 (KR) 10-2014-0173241

(51) Int. Cl. G01R 33/54; G01L 5/00; G01R 33/385; G01R 33/30; A61B 5/055; G01R 33/36

(52) U.S. Cl. CPC G01R 33/543; A61B 5/0555; G01R 33/3614; G01R 33/3852; G01R 33/30; G01L 5/00

(57) ABSTRACT

A magnetic resonance imaging (MRI) apparatus and a method of operating the MRI apparatus. The MRI apparatus includes: a monitoring unit configured to monitor an operation of the MRI apparatus; and a control unit configured to determine one of a plurality of power modes for the MRI apparatus based on the monitored operation and control the MRI apparatus to operate in the determined power mode.

### CLAIM OF PRIORITY

This application claims the benefit of priority from Korean Patent Application No. 10-2014-0173241, filed on Dec. 4, 2014, in the Korean Intellectual Property Office, the disclosure of which is incorporated by reference herein in its entirety.

### BACKGROUND

1. Field of the Disclosure

One or more exemplary embodiments relate to a magnetic resonance imaging (MRI) apparatus and a method of operating the MRI apparatus. More particularly, the present disclosure relates to an MRI apparatus that operates in one of a plurality of power modes for energy efficiency and a method of operating the MRI apparatus.

2. Description of the Related Art

A magnetic resonance imaging (MRI) apparatus uses a magnetic field to capture an internal image of a subject. An MRI apparatus is in widespread use for accurate diagnosis of diseases because it shows stereoscopic images of bones, lumbar discs, joints, nerve ligaments, the heart, brain tissue, etc., at desired angles.

Since the MRI apparatus generates a magnetic field by using a very strong electrical current, a huge amount of power is required to operate the MRI apparatus. There is an ongoing need to reduce the power usage of an MRI apparatus.

### SUMMARY

One or more exemplary embodiments of the present disclosure are related to an magnetic resonance imaging (MRI) apparatus and a method of operating the MRI apparatus that are capable of reducing power consumption by operating the MRI apparatus in a plurality of power modes. According to an exemplary embodiment, if the operation of an MRI apparatus is monitored and it is determined that the MRI apparatus does not require a high power consumption for operation of particular tasks, so the MRI apparatus may be set to operate in a low power mode.

Additional aspects will be set forth in part in the description which follows and, in part, will be apparent to a person of ordinary skill in the art reading the description, or may be learned by practice of the presented exemplary embodiments.

According to one or more exemplary embodiments, an MRI apparatus may include: a monitoring unit comprising hardware circuitry configured to monitor an MRI scanning operation of the MRI apparatus; and a control unit comprising hardware circuitry such as one or more processors or microprocessors configured to determine (i.e. identify), based on the monitored MRI scanning operation, an associated power mode out of a plurality of power modes for the MRI apparatus, control the MRI apparatus to operate based on the associated power mode, and change the associated power mode to another power mode based on a change in the monitored MRI scanning operation.

When an MRI scan is completed, the control unit may control the MRI apparatus to change the associated power mode so as to operate in a lower power mode. The lower power mode may be the lowest of the plurality of power modes, or may be a lower power mode than the associated power mode but sufficiently high to permit successful operation of tasks other than the scanning operation that just ended.

The control unit may control the MRI apparatus to operate in a standby mode prior to the MRI starting a scanning operation.

The control unit may be configured control the MRI apparatus to operate in a “quick start” mode when the MRI scanning operation begins.

The MRI apparatus may further include at least one of a patient table, a gradient amplifier, and an amplifier connected to a radio frequency (RF) coil, and the monitoring unit may monitor an operation of at least one of the patient table, the gradient amplifier, and the amplifier connected to the RF coil.

The control unit may control the MRI apparatus to operate in one of the plurality of power modes that is determined based on pressure detected via a sensor mounted on the patient table.

If a torque being applied to a motor of the table is less than a predetermined threshold, the control unit may control the MRI apparatus to operate in the low power mode.

If an operation of at least one of the gradient amplifier and the amplifier connected to the RF coil is not detected for a predetermined time, the control unit may control the MRI apparatus to operate in the low power mode because the scanning operation likely ended.

According to one or more exemplary embodiments, an MRI apparatus includes: a monitoring unit configured to monitor at least one operation of the MRI apparatus; a control unit configured to identify an associated power mode of a plurality of power modes for the MRI apparatus based on the operation being monitored, and to control the MRI apparatus to operate in the associated power mode; and an RF coil configured to be removably connected from at least one body part of an object. If the RF coil is disconnected from the at least one body part of the object, the control unit may control the MRI apparatus to operate in a low power mode.

According to one or more exemplary embodiments, an MRI apparatus includes: a monitoring unit configured to monitor an operation of the MRI apparatus; a control unit comprising hardware circuitry configured to identify an associated power mode out of a plurality of power modes for the MRI apparatus based on the monitored operation and control the MRI apparatus to operate in the associated power mode; and an input unit configured to receive a user input for selecting the associated power mode from among the plurality of power modes for operation of the MRI apparatus.

The input unit may receive a user input for inputting at least one of information about an object and information about scanning of the object, and the control unit may control the MRI apparatus to operate in a low power mode while the input unit is receiving the user input.

The control unit may be configured to control power by controlling a voltage that is supplied to a motor of a table included in the MRI apparatus.

The control unit may be configured to control an operation of at least one of a display and an audio system mounted on a gantry of the MRI apparatus.

The control unit may configured to control an operation of at least one of a gradient amplifier and an amplifier connected to an RF coil, the gradient amplifier and the amplifier being included in the MRI apparatus.

The control unit may be configured to control an operation of a heat exchanger included in the MRI apparatus.

According to one or more exemplary embodiments, a method of operating an MRI apparatus may include: monitoring an MRI scanning operation of the MRI apparatus; identifying an associated power mode based on the monitored MRI scanning operation from a plurality of power modes for the MRI apparatus; controlling the MRI apparatus to operate in the associated power mode; changing the associated power mode to another power mode based on a change in the monitored MRI scanning operation; and controlling the MRI apparatus to operate in a low power mode if an MRI scan is completed. The controlling of the MRI apparatus may include controlling the MRI apparatus to operate in a standby mode prior to the MRI starting a scanning operation.

The controlling of the MRI apparatus may further include controlling the MRI apparatus to operate in a quick start mode when the MRI scanning operation starts.

According to one or more exemplary embodiments, a method of operating an MRI apparatus includes: monitoring an operation of the MRI apparatus; determining a particular power mode out of a plurality of power modes for the MRI apparatus based on the monitored operation; controlling the MRI apparatus to operate in the particular power mode; and controlling the MRI apparatus according to a low power mode that is lower than the particular power mode if an RF coil configured to be attached to or detached from at least one body part of an object is disconnected from the at least one body part of the object.

According to one or more exemplary embodiments, a method of operating an MRI apparatus includes: monitoring an operation of the MRI apparatus; determining a particular power mode out of a plurality of power modes of operation for the MRI apparatus based on the monitored operation; controlling the MRI apparatus to operate in the particular power mode; and receiving a user input for selecting the determined power mode from among the plurality of power modes for the MRI apparatus.

According to the exemplary embodiments, the MRI apparatus may operate in different power modes according to a particular operation of the MRI apparatus, and thus power consumption by the MRI apparatus may be reduced. If it is determined that the operation of the MRI apparatus does not require high power consumption based on a result of monitoring the operation thereof, the MRI apparatus may enter a low power mode.

### BRIEF DESCRIPTION OF THE DRAWINGS

These and/or other aspects will become apparent and more readily appreciated by a person of ordinary skill in the art from the following description of the exemplary embodiments, taken in conjunction with the accompanying drawings in which:

FIG. 1 is a block diagram of a magnetic resonance imaging (MRI) apparatus according to an exemplary embodiment;

FIG. 2 is a block diagram of an MRI apparatus according to another exemplary embodiment;

FIG. 3 is a diagram illustrating processing of an MR image according to an exemplary embodiment;

FIG. 4 is a flowchart of a method of operating an MRI apparatus according to an exemplary embodiment;

FIG. 5 is a flowchart of a method of operating an MRI apparatus according to another exemplary embodiment;

FIG. 6 is a flowchart of a method of operating an MRI apparatus according to another exemplary embodiment;

FIG. 7 illustrates a screen for operating an MRI apparatus according to an exemplary embodiment;

FIG. 8 is a schematic diagram of a general MRI system; and

FIG. 9 illustrates a configuration of a communication unit.

### DETAILED DESCRIPTION

Advantages and features of one or more embodiments of the present disclosure and methods of accomplishing the same may be understood more readily by reference to the following detailed description of the embodiments and the accompanying drawings. In this regard, the present embodiments may have different forms and should not be construed as being limited to the descriptions set forth herein. Rather, these embodiments are provided so that this disclosure will be thorough in fully conveying the concept of the present embodiments to one of ordinary skill in the art, and the present disclosure will only be defined by the appended claims.

Terms used herein will now be briefly described and then one or more embodiments of the present disclosure will be described in detail.

All terms including descriptive or technical terms which are used herein should be construed as having meanings that are understood by one of ordinary skill in the art. However, the terms may have different meanings according to the intention of one of ordinary skill in the art, precedent cases, or the appearance of new technologies. Also, some terms may be arbitrarily selected by the applicant, and in this case, the meaning of the selected terms will be described in detail in the detailed description of the disclosure. Thus, the terms used herein have to be defined based on the meaning of the terms together with the description throughout the specification as a person of ordinary skill in the art would understand them.

When a part “includes” or “comprises” an element, unless there is a particular description contrary thereto, the part can further include other elements, and does not exclude the other elements. Also, the term “unit” in the embodiments of the present disclosure means a software component or hardware component such as a field-programmable gate array (FPGA) or an application-specific integrated circuit (ASIC), and performs a specific function. The unit is a statutory element under its broadest reasonable interpretation herein, and may be formed so as to be in an addressable storage medium, or may be formed so as to operate one or more processors. Thus, for example, the term “unit” is in all cases a statutory element, may include components such as software components, object-oriented software components, class components, and task components, and may include processes, functions, attributes, procedures, subroutines, segments of program code, drivers, firmware, micro codes, circuits, data, a database, data structures, tables, arrays, or variables and all software of firmware is loaded into hardware for execution. A function provided by the components and units may be associated with the smaller number of components and units, or may be divided into additional components and “units”.

Reference will now be made in detail to embodiments, examples of which are illustrated in the accompanying drawings. In the following description, well-known functions or constructions may not be described in detail so as not to obscure appreciation of the disclosure by a person of ordinary skill in the art with unnecessary detail about well-known functions and structures.

In the present specification, an “image” may refer to multi-dimensional data composed of discrete image elements (e.g., pixels in a two-dimensional (2D) image and voxels in a three-dimensional (3D) image). For example, the image may be a medical image of an object captured by an X-ray apparatus, a computed tomography (CT) apparatus, a magnetic resonance imaging (MRI) apparatus, an ultrasound diagnosis apparatus, or another medical imaging apparatus.

Furthermore, in the present specification, an “object” may be a human, an animal, or a part of a human or animal. For example, the object may be an organ (e.g., the liver, the heart, the womb, the brain, a breast, or the abdomen, just to name some non-limiting examples), a blood vessel, or a combination thereof. Furthermore, the “object” may be a phantom. The phantom means a material having a density, an effective atomic number, and a volume that are approximately the same as those of an organism. For example, the phantom may be a spherical phantom having properties similar to the human body.

Furthermore, in the present specification, a “user” may be, but is not limited to, a medical expert, such as a medical doctor, a nurse, a medical laboratory technologist, or a technician who repairs a medical apparatus.

Furthermore, in the present specification, an “MR image” refers to an image of an object obtained by using the nuclear magnetic resonance principle.

Furthermore, in the present specification, a “pulse sequence” refers to continuity of signals repeatedly applied by an MRI apparatus. The pulse sequence may include a time parameter of a radio frequency (RF) pulse, for example, repetition time (TR) or echo time (TE).

Furthermore, in the present specification, a “pulse sequence schematic diagram” shows an order of events that occur in an MRI apparatus. For example, the pulse sequence schematic diagram may be a diagram showing an RF pulse, a gradient magnetic field, an MR signal, or the like according to time.

An MRI system is an apparatus for acquiring a sectional image of a part of an object by expressing, in a contrast comparison, a strength of an MR signal with respect to a radio frequency (RF) signal generated in a magnetic field having a specific strength. For example, if an RF signal that only resonates a specific atomic nucleus (for example, a hydrogen atomic nucleus) is emitted for an instant toward the object placed in a strong magnetic field and then such emission stops, an MR signal is emitted from the specific atomic nucleus, and thus the MRI system may receive the MR signal and acquire an MR image. The MR signal denotes an RF signal emitted from the object. An intensity of the MR signal may be determined according to a density of a predetermined atom (for example, hydrogen) of the object, a relaxation time T1, a relaxation time T2, and a flow of blood or the like.

MRI systems include characteristics different from those of other imaging apparatuses. Unlike imaging apparatuses such as CT apparatuses that acquire images according to a direction of detection hardware, MRI systems may acquire 2D images or 3D volume images that are oriented toward an optional point. MRI systems do not expose objects or examiners to radiation, unlike CT apparatuses, X-ray apparatuses, position emission tomography (PET) apparatuses, and single photon emission CT (SPECT) apparatuses, may acquire images having high soft tissue contrast, and may acquire neurological images, intravascular images, musculoskeletal images, and oncologic images that are required to precisely capturing abnormal tissues.

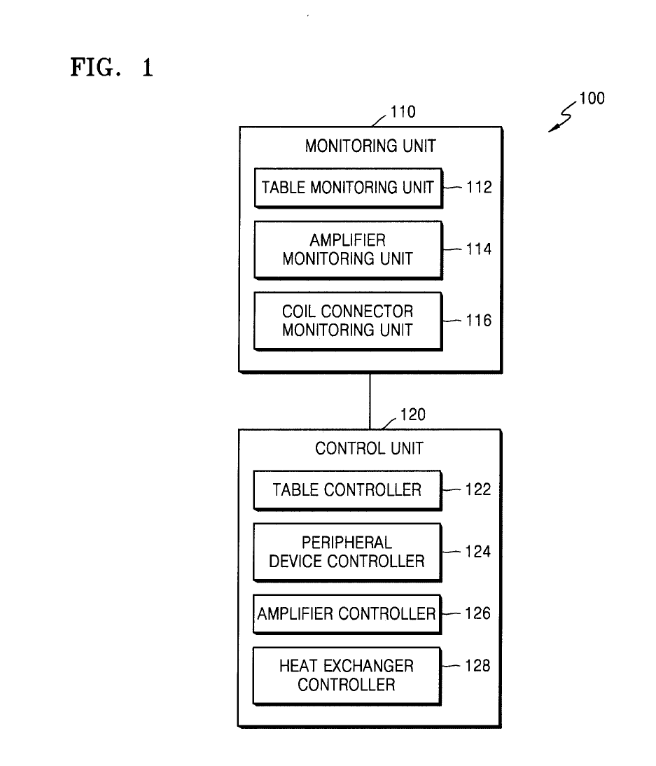

FIG. 1 is a block diagram of a magnetic resonance imaging (MRI) apparatus 100 according to an exemplary embodiment.

Referring now to FIG. 1, the MRI apparatus 100 according to the present exemplary embodiment may be a device that performs an MRI scan of an object and processes an image acquired using the MRI scan. The MRI apparatus 100 may include a monitoring unit 110 and a control unit 120. Both the monitoring unit and control unit, and units contained therein, comprises hardware circuitry configured for operation, and do not constitute software per se or pure software.

According to an exemplary embodiment, the monitoring unit 110 may monitor an operation of the MRI apparatus 100. For example, the monitoring unit 110 may monitor the operation of the MRI apparatus 100 by monitoring a patient table, a gradient amplifier, a radio frequency (RF) amplifier, a connector of a removable RF coil, a transmission and reception switch, an RF transmitter, an RF receiver, etc. The operation of the MRI apparatus 100 may include a scanning operation. The scanning operation may include not only an MRI scanning operation but also a preparatory operation related to an MRI scan. For example, the scanning operation may include a preparatory operation performed prior to the MRI scan, a standby operation and a start operation related to a next MRI scan, etc.

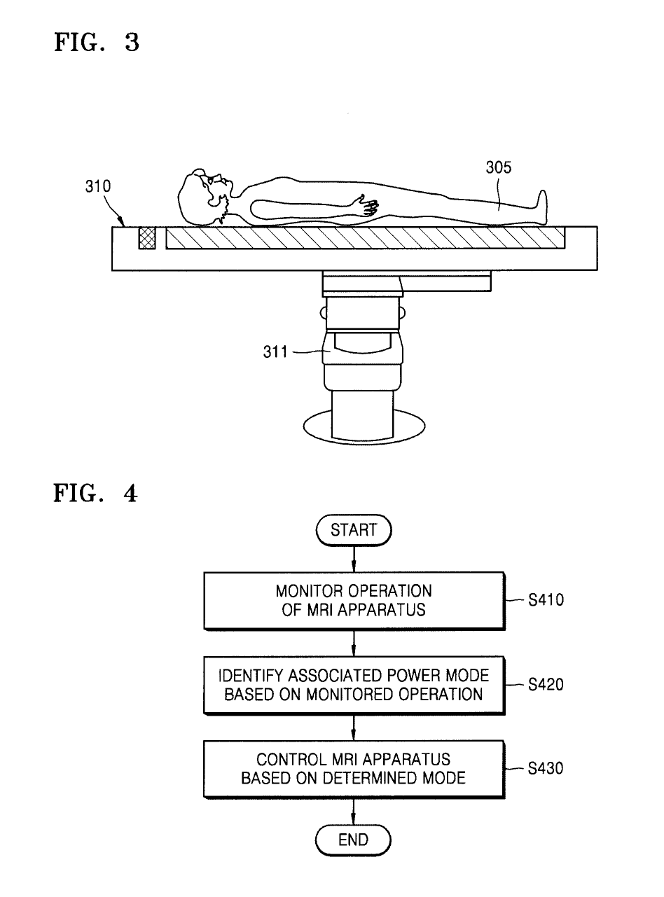

According to an exemplary embodiment, the monitoring unit 110 may include a table monitoring unit 112, an amplifier monitoring unit 114, a coil connector monitoring unit 116. Each of these units comprise hardware such as circuitry configured for operation, The table monitoring unit 112 may monitor a state of a table (a table 310 of FIG. 3). To do so, the table monitoring unit 112 may measure pressure being applied to the table 310 by using a sensor (311 of FIG. 3) mounted on the table 310.

FIG. 3 shows that an object 305 lies on the patient table 310 where the sensor 311 is disposed. For convenience of explanation, other components of the MRI apparatus are not shown in FIG. 3. The sensor 311 may be disposed on the outside of the table 310 or built into the table 310.

According to an exemplary embodiment, the sensor 311 may be a sensor for measuring pressure being applied to the table 310 due to a weight of the object 305, in this case a patient. If the sensor 311 measures pressure being applied to the table 311, the sensor 311 may measure at least one of a magnitude of the pressure and a range of a region where the pressure is applied.

For example, if the sensor 311 detects that the pressure being applied to the table 310 has a magnitude greater than a predetermined magnitude, the table monitoring unit 112 may determine that a phantom has been placed on the table 310.

As another example, if the sensor 311 detects that pressure is applied to a region including a predetermined region on the table 310, the table monitoring unit 112 may determine that a patient's body lies completely on the table 310. If the sensor 311 detects that the pressure is applied to a portion of a predetermined region on the table 310, the table monitoring unit 112 may determine that a phantom has been placed on the table 300.

According to another exemplary embodiment, the sensor 311 may be a sensor for measuring a torque being applied to a motor (not shown) of the table 310. The sensor 311 may be a sensor built into the table 310. The table monitoring unit 112 may monitor a state of the table 310 by using a torque being applied to the motor of the table 310.

According to another exemplary embodiment, the sensor 311 may be a sensor for measuring both pressure being applied to the table 310 due to a weight of the object 305 and torque being applied to the motor of the table 310. The table monitoring unit 112 may monitor the weight of the object 305 placed on the table 310, a moving speed of the table 310, etc., based on a result of measurement by the sensor 311.

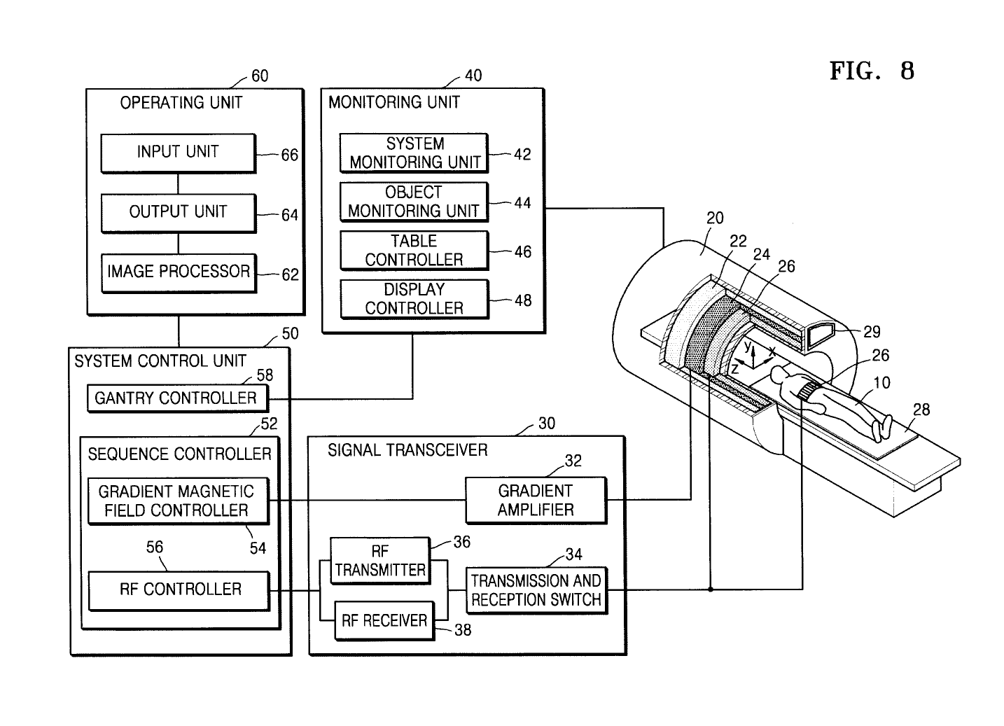

The amplifier monitoring unit 114 may monitor signals of a gradient amplifier and an RF amplifier. The gradient amplifier may control a pulse signal provided to a gradient coil (24 of FIG. 8). The RF amplifier may control an RF pulse supplied for driving an RF coil (26 of FIG. 8). The operation of the MRI apparatus 100 may be monitored by the monitored signals of the gradient amplifier and the RF amplifier. For example, if the signals of the gradient amplifier and the RF amplifier are not detected for a predetermined time, it may be determined that a scanning operation ceases to be performed by the MRI apparatus 100.

For example, the coil connector monitoring unit 116 may monitor a state of connection of a plurality of removable RF coils. The plurality of removable RF coils may include RF coils for parts of the object 305 including a head RF coil, a breast RF coil, a leg RF coil, a neck RF coil, a shoulder RF coil, a wrist RF coil, an ankle RF coil, etc. According to an exemplary embodiment, the coil connector monitoring unit 116 may detect coils that are in a state of being connected to and disconnected from body parts, among the plurality of removable RF coils that can be attached to or detached from the body parts. For example, the coil connector monitoring unit 116 may detect wrist and ankle RF coils as being disconnected from the wrist and ankle, respectively, among RF coils that are connected to body parts of an object

The above-described components of the monitoring unit 110 are merely an example of components for monitoring the operation of the MRI apparatus 100, and the configuration of the monitoring unit 110 is not limited thereto the example shown and described.

According to an exemplary embodiment, the control unit 120 may identify an associated power mode from a plurality of power modes for the MRI apparatus 100 based on the operation of the MRI apparatus 100 monitored by the monitoring unit 110.

The plurality of power modes may include a normal power mode in which power consumption by the MRI apparatus 100 is not controlled and a low power mode in which power consumption by at least one of the components of the MRI apparatus 100 is controlled.

The control unit 120 may identify a power mode based on various operations monitored by the monitoring unit 110.

For example, if it is determined, based on a result of monitoring by the table monitoring unit 112, that an MRI scan of a human body is being performed, the control unit 120 may identify a power mode for the MRI apparatus as a normal power mode and control the MRI apparatus 100 to operate in the normal power mode. If an object is determined, based on a result of monitoring by the table monitoring unit 112, to be a phantom that is an object designed for study, the control unit 120 may identify a power mode for the MRI apparatus 100 as a low power mode and control the MRI apparatus 100 to operate in the low power mode.

As another example, if the amplifier monitoring unit 114 does not detect signals of the gradient amplifier and the RF amplifier for a predetermined time and thus determines that a scanning operation ceases to be performed by the MRI apparatus 100, the control unit 120 may identify a power mode for the MRI apparatus 100 as a low power mode and control the MRI apparatus 100 to operate in the low power mode.

As another example, the coil connector monitoring unit 116 may detect a head RF coil, a breast RF coil, a leg RF coil, a neck RF coil, a shoulder RF coil, a wrist RF coil, and an ankle RF coil as being normally connected to their corresponding body parts. In this case, the control unit 120 may control the MRI apparatus 100 to operate in a normal power mode. If the coil connector monitoring unit 116 detects the leg RF coil, the wrist RF coil, and the ankle RF coil as being disconnected from their corresponding body parts, the control unit 120 may control the MRI apparatus 100 to operate in a low power mode.

According to an exemplary embodiment, a low power mode may include a low power mode for each of the components of the MRI apparatus 100. For example, the low power mode may include low power modes for a table system, a peripheral device, an amplifier, and a heat exchanger.

The low power mode may include a standby mode as a scanning operation changes. The standby mode is a mode for quickly starting an MRI scan in a state where an MRI scanning operation is not being performed. In the standby mode, a table, peripheral devices, an amplifier, and a heat exchanger of the MRI apparatus 100 may be operated to prepare for an MRI scan. When the MRI apparatus 100 is in the standby mode, the MRI apparatus 100 may consume a smaller amount of power than normal power. The MRI apparatus 100 that is in the standby mode may enter a quick start mode to initiate the MRI scanning operation. The quick start mode may be a mode in which normal power is consumed without limitation in order to quickly start the MRI scanning operation.

According to an exemplary embodiment, the control unit 120 may control the MRI apparatus 100 based on a identified power mode. The control unit 120 may include a table controller 122, a peripheral device controller 124, an amplifier controller 126, and a heat exchanger controller 128.

The table controller 122 may limit at least one of a voltage and a current that are supplied to a table motor (not shown) to be less than or equal to a predetermined value, based on the identifed power mode. In detail, if fast scanning of an object is not required, the table controller 122 may limit power that is supplied to the table motor to low power. For example, the table controller 122 may control a table to operate in a low power mode by limiting the amount of torque applied to the table motor during acceleration and deceleration intervals. As described above, the table controller 122 may control the MRI apparatus 100 to operate in a low power mode for the table by controlling a system of the table.

The peripheral device controller 124 may control the peripheral devices based on a identified power mode. The peripheral devices may include devices other than devices directly used for an MRI scan and may include a device for a patient, mounted inside or outside a gantry. For example, the peripheral device controller 124 may block supply of power to at least one of an in bore display for a patient within the gantry, an audio system, a noise cancellation system, and a ventilator mounted on the inside of the gantry, and a display mounted on the outside of the gantry. The peripheral device controller 124 may control the MRI apparatus 100 to operate in a low power mode for the peripheral devices by blocking supply of power to at least one of the peripheral devices

The amplifier controller 126 may limit at least one of a voltage and a current that are supplied to the amplifier to less than or equal to a predetermined value, based on the identified power mode. For example, the amplifier controller 126 may reduce consumption of power that is regularly supplied to a gradient amplifier and an RF coil amplifier by limiting output values of the gradient amplifier and the RF coil amplifier to be less than or equal to a predetermined value. The amplifier controller 126 may control the MRI apparatus 100 to operate in a low power mode for the amplifier by limiting power supplied to the amplifier to be less than or equal to predetermined power.

The heat exchanger controller 128 may limit power supplied to the heat exchanger based on the determined power mode. The heat exchanger may be a system for withdrawing heat by using a flow of coolant. The heat exchanger may control a flow of heat generated in the MRI apparatus 100 by controlling the coolant to maintain a constant velocity. A large amount of power may be consumed by a compressor of the heat exchanger. The heat exchanger controller 126 may control the MRI apparatus 100 to operate in a low power mode for the heat exchanger by setting a critical temperature of the MRI apparatus 100 to a high temperature and maintaining a coolant velocity at a low velocity.

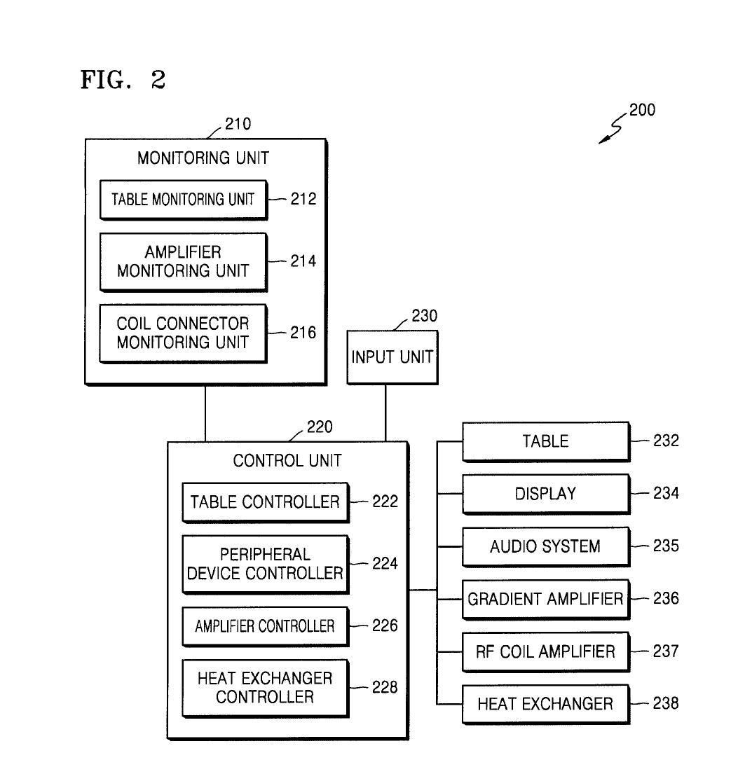

FIG. 2 is a block diagram of an MRI apparatus 200 according to another exemplary embodiment.

Referring to FIG. 2, The MRI apparatus 200 according to the present exemplary embodiment may include a monitoring unit 210, a control unit 220, and an input unit 230. Since the operation and detailed configurations of the monitoring unit 210 and the control unit 220 correspond to those of the monitoring unit 110 and the control unit 120 shown in FIG. 1, descriptions that are already provided above with respect to FIG. 1 will not be repeated below.

Unlike in the MRI apparatus 100, the MRI apparatus 200 may further include the input unit 230.

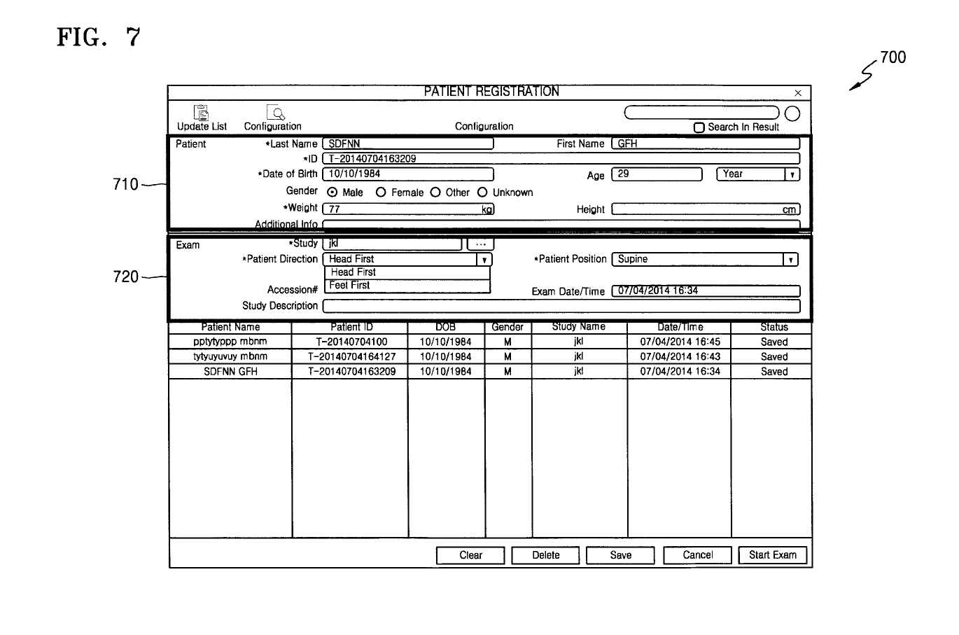

The input unit 230 may be a unit via which the user inputs data necessary for controlling the MRI apparatus 200. According to an exemplary embodiment, the input unit 230 may receive a user input for selecting one of a plurality of power modes for the MRI apparatus 200. Furthermore, the input unit 230 may receive a user input for inputting at least one of information about an object and information about scanning of the object. The user may view information about scanning of the object input via a displayed screen (700 of FIG. 7)

The control unit 220 may control the MRI apparatus 200 to operate in a low power mode while the input unit 230 is receiving a user input.

The MRI apparatus 200 may further include a table 232 controlled by the control unit 220, displays mounted inside and outside a gantry, an audio system 235 mounted inside the gantry, a gradient amplifier 236, an RF coil amplifier 237, and a heat exchanger 238. Since these have been described above with respect to FIG. 1, the descriptions thereof will not be repeated below.

FIG. 4 is a flowchart of a method of operating the MRI apparatus 100 (200) according to an exemplary embodiment. The method according to the present exemplary embodiment may be performed by the MRI apparatus 100 (200).

Referring to FIG. 4, the MRI apparatus 100 (200) may monitor an operation thereof (S410). To do so, the MRI apparatus 100 (200) may monitor a table, a gradient amplifier, an RF amplifier, connectors of a removable RF coil, a transmission and reception switch, an RF transmitter, an RF receiver, etc. In detail, the MRI apparatus 100 (200) may monitor a state of the table by measuring pressure being applied to the table and torque being applied to a motor of the table. Furthermore, the MRI apparatus 100 (200) may monitor signals of the gradient amplifier and the RF amplifier and a connection of the connectors of a removable RF coil.

The MRI apparatus 100 (200) may identify an associated power mode from a plurality of power modes based on the operation monitored in operation S410 (S420). The plurality of power modes may include a normal power mode in which power consumption by the MRI apparatus 100 (200) is not controlled and a low power mode in which power consumption by at least one of the components of the MRI apparatus 100 (200) is controlled.

The MRI apparatus 100 (200) may control the components thereof based on the associated power mode identified in operation S420. The MRI apparatus 100 (200) may control the table, peripheral devices, an amplifier, a heat exchanger, etc.

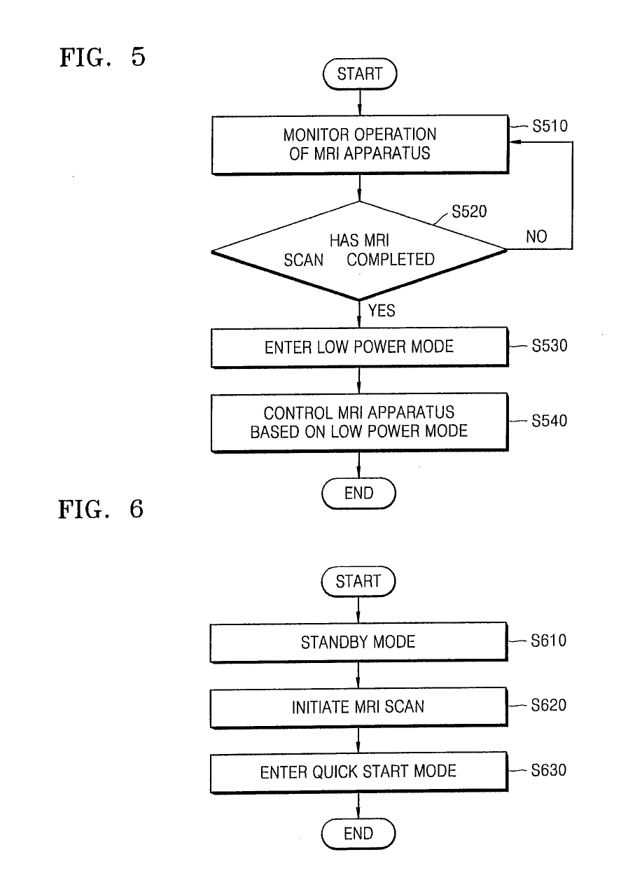

FIG. 5 is a flowchart of a method of operating the MRI apparatus 100 (200) according to another exemplary embodiment.

Referring now to FIG. 5, the MRI apparatus 100 (200) may monitor an operation of the MRI apparatus 100 (200) (S510).

The MRI apparatus 100 (200) may determine whether an MRI scan is completed based on the operation monitored in operation S510 (S520).

In detail, the MRI apparatus 100 (200) may determine whether an MRI scan is completed based on a detected status of operations of a table, a gradient amplifier, an RF amplifier, etc. For example, if the operation of the table or signals of the gradient amplifier and the RF amplifier are not detected during a preset standby time, the MRI apparatus 100 (200) may determine that the MRI scan is completed. As another example, if a time input in advance via the input unit 230 elapses (e.g., PM23:00 and PM24:00), the MRI apparatus 100 (200) may determine that the MRI scan is completed. As another example, the user may input completion of the MRI scan for a patient via the input unit 230.

If it is determined that the MRI scan is completed in operation S520, the MRI apparatus 100 (200) may enter a low power mode. If it is determined that the MRI scan is not completed in operation S520, the MRI apparatus 100 (200) may return to operation S510.

The MRI apparatus 100 (200) may control itself to operate in the low power mode (S540).

FIG. 6 is a flowchart of a method of operating the MRI apparatus 100 according to another exemplary embodiment.

With reference to FIG. 6, while an MRI scanning operation is not being performed, the MRI apparatus 100 (200) may operate in a standby mode (S610). The standby mode is a mode for quickly starting an MRI scan in a state where the MRI scanning operation is not being performed. When the MRI apparatus 100 (200) is in the standby mode, the MRI apparatus 100 may consume a smaller amount of power than normal power to prepare for an MRI scan.

If the MRI scan starts in the MRI apparatus 100 (200) (S620), the MRI apparatus 100 (200) may enter a quick start mode (S630). According to the quick start mode, normal power may be consumed when an MRI scanning operation starts to be performed.

According to the method of FIG. 6, the MRI apparatus 100 (200) may quickly initiate a scanning operation by changing the standby mode to the quick start mode.

FIG. 7 illustrates the screen 700 for operating the MRI apparatus according to an exemplary embodiment.

The screen 700 according to the present exemplary embodiment may show information about scanning of an object. The user may enter information about the object and the information about scanning of the object via the input unit 230. The input information may be displayed on first and second regions 710 and 720 of the screen 700. For example, the information about the object may include a patient's last name, first name, identification (ID), date of birth, gender, age, weight, height, etc. The information about the object may be displayed on the first region 710 of the screen 700. Furthermore, for example, the information about scanning of the object may include a patient direction indicating a body part that enters a gantry first from among the object's body parts, a patient position indicating an object's pose, etc. The information about scanning of the object may be displayed on the second region 720 of the screen 700.

FIG. 8 is a block diagram of a general MRI system. Referring now to FIG. 8, the general MRI system may include a gantry 20, a signal transceiver 30, a monitoring unit 40, a system control unit 50, and an operating unit 60.

The gantry 20 prevents external emission of electromagnetic waves generated by a main magnet 22, a gradient coil 24, and an RF coil 26. A magnetostatic field and a gradient magnetic field are formed in a bore in the gantry 20, and an RF signal is emitted toward an object 10.

The main magnet 22, the gradient coil 24, and the RF coil 26 may be arranged in a predetermined direction of the gantry 20. The predetermined direction may be a coaxial cylinder direction. The object 10 may be disposed on a table 28 that is capable of being inserted into a cylinder along a horizontal axis of the cylinder.

The main magnet 22 generates a magnetostatic field or a static magnetic field for aligning magnetic dipole moments of atomic nuclei of the object 10 in a constant direction. A precise and accurate MR image of the object 10 may be obtained due to a magnetic field generated by the main magnet 22 being strong and uniform.

The gradient coil 24 includes X, Y, and Z coils for generating gradient magnetic fields in X-, Y-, and Z-axis directions crossing each other at right angles. The gradient coil 24 may provide location information of each region of the object 10 by differently inducing resonance frequencies according to the regions of the object 10.

The RF coil 26 may emit an RF signal toward a patient and receive an MR signal emitted from the patient. In detail, the RF coil 26 may transmit, toward atomic nuclei included in the patient and having precessional motion, an RF signal having the same frequency as that of the precessional motion, stop transmitting the RF signal, and then receive an MR signal emitted from the atomic nuclei included in the patient.

For example, in order to transit an atomic nucleus from a low energy state to a high energy state, the RF coil 26 may generate and apply an electromagnetic wave signal that is an RF signal corresponding to a type of the atomic nucleus, to the object 10. When the electromagnetic wave signal generated by the RF coil 26 is applied to the atomic nucleus, the atomic nucleus may transit from the low energy state to the high energy state. Then, when electromagnetic waves generated by the RF coil 26 disappear, the atomic nucleus to which the electromagnetic waves were applied transits from the high energy state to the low energy state, thereby emitting electromagnetic waves having a Lamor frequency. In other words, when the applying of the electromagnetic wave signal to the atomic nucleus is stopped, an energy level of the atomic nucleus is changed from a high energy level to a low energy level, and thus the atomic nucleus may emit electromagnetic waves having a Lamor frequency. The RF coil 26 may receive electromagnetic wave signals from atomic nuclei included in the object 10.

The RF coil 26 may be realized as one RF transmitting and receiving coil having both a function of generating electromagnetic waves each having an RF that corresponds to a type of an atomic nucleus and a function of receiving electromagnetic waves emitted from an atomic nucleus. Alternatively, the RF coil 26 may be realized as a transmission RF coil having a function of generating electromagnetic waves each having an RF that corresponds to a type of an atomic nucleus, and a reception RF coil having a function of receiving electromagnetic waves emitted from an atomic nucleus.

The RF coil 26 may be fixed to the gantry 20 or may be detachable. When the RF coil 26 is detachable, the RF coil 26 may be an RF coil for a part of the object, such as a head RF coil, a chest RF coil, a leg RF coil, a neck RF coil, a shoulder RF coil, a wrist RF coil, or an ankle RF coil.

The RF coil 26 may communicate with an external apparatus via wires and/or wirelessly, and may also perform dual tune communication according to a communication frequency band.

The RF coil 26 may be a birdcage coil, a surface coil, or a transverse electromagnetic (TEM) coil according to structures.

The RF coil 26 may be a transmission exclusive coil, a reception exclusive coil, or a transmission and reception coil according to methods of transmitting and receiving an RF signal.

The RF coil 26 may be an RF coil having various numbers of channels, such as 16 channels, 32 channels, 72 channels, and 144 channels.

The gantry 20 may further include a display 29 disposed outside the gantry 20 and a display (not shown) disposed inside the gantry 20. The gantry 20 may provide predetermined information to the user or the object 10 through the display 29 and the display respectively disposed outside and inside the gantry 20.

The signal transceiver 30 may control the gradient magnetic field formed inside the gantry 20, i.e., in the bore, according to a predetermined MR sequence, and control transmission and reception of an RF signal and an MR signal.

The signal transceiver 30 may include a gradient amplifier 32, a transmission and reception switch 34, an RF transmitter 36, and an RF receiver 38.

The gradient amplifier 32 drives the gradient coil 24 included in the gantry 20, and may supply a pulse signal for generating a gradient magnetic field to the gradient coil 24 under the control of a gradient magnetic field controller 54. By controlling the pulse signal supplied from the gradient amplifier 32 to the gradient coil 24, gradient magnetic fields in X-, Y-, and Z-axis directions may be synthesized.

The RF transmitter 36 and the RF receiver 38 may drive the RF coil 26. The RF transmitter 36 may supply an RF pulse in a Lamor frequency to the RF coil 26, and the RF receiver 38 may receive an MR signal received by the RF coil 26.

The transmission and reception switch 34 may adjust transmitting and receiving directions of the RF signal and the MR signal. For example, the transmission and reception switch 34 may emit the RF signal toward the object 10 through the RF coil 26 during a transmission mode, and receive the MR signal from the object 10 through the RF coil 26 during a reception mode. The transmission and reception switch 34 may be controlled by a control signal output by an RF controller 56.

The monitoring unit 40 may monitor the gantry 20 or devices mounted on the gantry 20. The monitoring unit 40 may include a system monitoring unit 42, and an object monitoring unit 44.

The system monitoring unit 42 may monitor, for example, a state of the magnetostatic field, a state of the gradient magnetic field, a state of the RF signal, a state of the RF coil 26, a state of the table 28, a state of a device measuring body information of the object 10, a power supply state, a state of a thermal exchanger, and a state of a compressor.

The object monitoring unit 44 monitors a state of the object 10. In detail, the object monitoring unit 44 may include a camera for observing a movement or position of the object 10, a respiration measurer for measuring the respiration of the object 10, an electrocardiogram (ECG) measurer for measuring the electrical activity of the object 10, or a temperature measurer for measuring a temperature of the object 10.

The table controller 46 controls a movement of the table 28 where the object 10 is positioned. The table controller 46 may control the movement of the table 28 according to a sequence control of a sequence controller 52. For example, during moving imaging of the object 10, the table controller 46 may continuously or discontinuously move the table 28 according to the sequence control of the sequence controller 52, and thus the object 10 may be photographed in a field of view (FOV) larger than that of the gantry 20.

The display controller 48 controls the display 29 disposed outside the gantry 20 and the display disposed inside the gantry 20. In detail, the display controller 48 may control the display 29 and the display to be on or off, and may control a screen image to be output on the display 29 and the display. Also, when a speaker is located inside or outside the gantry 20, the display controller 48 may control the speaker to be on or off, or may control sound to be output via the speaker.

The system control unit 50 may include the sequence controller 52 for controlling a sequence of signals formed in the gantry 20, and a gantry controller 58 for controlling the gantry 20 and the devices mounted on the gantry 20.

The sequence controller 52 may include the gradient magnetic field controller 54 for controlling the gradient amplifier 32, and the RF controller 56 for controlling the RF transmitter 36, the RF receiver 38, and the transmission and reception switch 34. The sequence controller 52 may control, for example, the gradient amplifier 32, the RF transmitter 36, the RF receiver 38, and the transmission and reception switch 34 according to a pulse sequence received from the operating unit 60. Here, the pulse sequence includes all information required to control the gradient amplifier 32, the RF transmitter 36, the RF receiver 38, and the transmission and reception switch 34. For example, the pulse sequence may include information about a strength, an application time, and application timing of a pulse signal applied to the gradient coil 24.

The operating unit 60 may request the system control unit 50 to transmit pulse sequence information while controlling an overall operation of the MRI system.

The operating unit 60 may include an image processor 62 for receiving and processing the MR signal received by the RF receiver 38, an output unit 64, and an input unit 66.

The image processor 62 may process the MR signal received from the RF receiver 38 so as to generate MR image data of the object 10.

The image processor 62 receives the MR signal received by the RF receiver 38 and performs any one of various signal processes, such as amplification, frequency transformation, phase detection, low frequency amplification, and filtering, on the received MR signal.

The image processor 62, which includes hardware circuitry configured for operation, may arrange data in a k space (for example, also referred to as a Fourier space or a frequency space) of a memory, and rearrange the digital data into image data via 2D or 3D Fourier transformation.

The image processor 62 may perform a composition process or difference calculation process on image data if required. The composition process may include an addition process on a pixel or a maximum intensity projection (MIP) process. The image processor 62 may store not only the rearranged image data but also image data on which a composition process or a difference calculation process is performed, in a memory or an external server.

The image processor 62 may perform any of the signal processes on the MR signal in parallel. For example, the image processor 62 may perform a signal process on a plurality of MR signals received by a multi-channel RF coil in parallel so as to rearrange the plurality of MR signals into image data.

The output unit 64 may output image data generated or rearranged by the image processor 62 to the user. The output unit 64 may also output information required for the user to manipulate the MRI system, such as a user interface (UI), user information, or object information. Examples of the output units 64 may include a speaker, a printer, a cathode ray tube (CRT) display, a liquid crystal display (LCD), a plasma display panel (PDP), an organic light emitting diode (OLED) display, a field emission display (FED), a light emitting diode (LED) display, a vacuum fluorescent display (VFD), a digital light processing (DLP) display, a flat panel display (PFD), a three-dimensional (3D) display, a transparent display, and other various output devices well known to one of ordinary skill in the art, just to name a few non-limiting examples.

The user may input object information, parameter information, a scan condition, a pulse sequence, or information about image composition or difference calculation by using the input unit 66. The input unit 66 may be, for example, a keyboard, a mouse, a track ball, a voice recognizer, a gesture recognizer, a touch screen, or any one of other various input devices that are well known to one of ordinary skill in the art.

The signal transceiver 30, the monitoring unit 40, the system control unit 50, and the operating unit 60 are separate components in FIG. 8, but respective functions of the signal transceiver 30, the monitoring unit 40, the system control unit 50, and the operating unit 60 may be performed by another component. For example, the image processor 62 converts the MR signal received from the RF receiver 38 into a digital signal in FIG. 1, but alternatively, the conversion of the MR signal into the digital signal may be performed by the RF receiver 38 or the RF coil 26.

The gantry 20, the RF coil 26, the signal transceiver 30, the monitoring unit 40, the system control unit 50, and the operating unit 60 may be connected to each other by wire or wirelessly, and when they are connected wirelessly, the MRI system may further include an apparatus (not shown) for synchronizing clock signals therebetween. Communication between the gantry 20, the RF coil 26, the signal transceiver 30, the monitoring unit 40, the system control unit 50, and the operating unit 60 may be performed by using a high-speed digital interface, such as low voltage differential signaling (LVDS), asynchronous serial communication, such as a universal asynchronous receiver transmitter (UART), a low-delay network protocol, such as error synchronous serial communication or a controller area network (CAN), or optical communication.

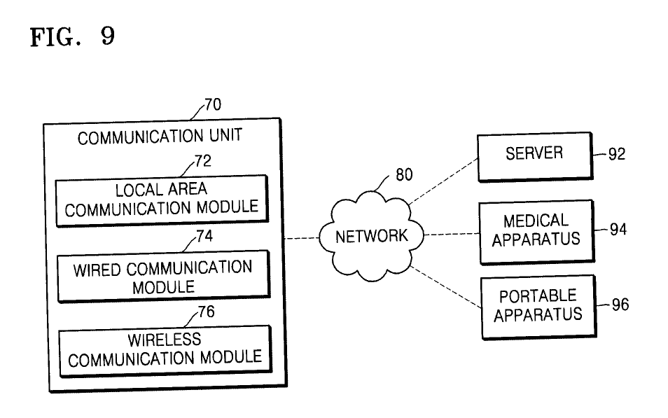

The MRI apparatus 100 (200) of FIG.1 (2) may be the external server 92, medical apparatus 94, or portable device 96 connected to an MRI system. In other words, the MRI apparatus 100 (200) may be connected to the communication unit 70 shown in FIG. 9 for operation thereof.

The apparatuses and methods of the disclosure can be implemented in hardware, and in part as firmware or via the execution of software or computer code in conjunction with hardware that is stored on a non-transitory machine readable medium such as a CD ROM, a RAM, a floppy disk, a hard disk, or a magneto-optical disk, or computer code downloaded over a network originally stored on a remote recording medium or a non-transitory machine readable medium and stored on a local non-transitory recording medium for execution by hardware such as a processor, so that the methods described herein are loaded into hardware such as a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc., that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein. In addition, an artisan understands and appreciates that a “processor”, “microprocessor” “controller”, or “control unit” constitute hardware in the claimed disclosure that contain circuitry that is configured for operation. Under the broadest reasonable interpretation, the appended claims constitute statutory subject matter in compliance with 35 U.S.C. §101 and none of the elements are software per se.

The definition of the terms “unit” or “module” as referred to herein are to be understood as constituting hardware circuitry such as a CCD, CMOS, SoC, AISC, FPGA, a processor or microprocessor (a controller) configured for a certain desired functionality, or a communication module containing hardware such as transmitter, receiver or transceiver, or a non-transitory medium comprising machine executable code that is loaded into and executed by hardware for operation, in accordance with statutory subject matter under 35 U.S.C. §101 and do not constitute software per se.

The embodiments of the present disclosure may be written as computer programs and may be implemented in general-use digital computers that execute the programs using a computer-readable recording medium.

Examples of the computer-readable recording medium include magnetic storage media (e.g., ROM, floppy disks, hard disks, etc.), optical recording media (e.g., CD-ROMs or DVDs), etc.

While the present disclosure has been particularly shown and described with reference to exemplary embodiments thereof, it will be understood by those of ordinary skill in the art that various changes in form and details may be made therein without departing from the spirit and scope of the present disclosure as defined by the following claims. Accordingly, the above embodiments and all aspects thereof are examples only and are not limiting.

What is claimed is:

1. A magnetic resonance imaging (MRI) apparatus comprising:
a monitoring unit configured to monitor a status of an MRI scanning operation of the MRI apparatus; and
a control unit configured to identify an associated power mode from a plurality of power modes for operating the MRI apparatus based on the monitored MRI scanning operation, control the MRI apparatus based on the associated power mode, and change the associated power mode to another power mode based on a change in the monitored MRI scanning operation,
wherein, in response to the monitored MRI scanning operation being completed, the control unit controls the MRI apparatus to operate in a low power mode that consumes less power than the associated power mode.

2. The MRI apparatus of claim 1, wherein the control unit controls the MRI apparatus to operate in a standby mode prior to starting the MRI scanning operation.

3. The MRI apparatus of claim 2, wherein the control unit controls the MRI apparatus to operate in a quick start mode when the MRI scanning operation starts.

4. The MRI apparatus of claim 1, further comprising at least one of a patient table, a gradient amplifier, a radio frequency (RF) coil and an amplifier connected to the RF coil,
wherein the monitoring unit monitors an operation of at least one of the patient table, the gradient amplifier, and the amplifier connected to the RF coil.

5. The MRI apparatus of claim 4, wherein the control unit controls the MRI apparatus to operate in one of the plurality of power modes based on a pressure detected by a sensor mounted on the patient table.

6. The MRI apparatus of claim 5, wherein, when the control unit identifies a value of torque being applied to a motor of the patient table is less than a threshold, the control unit controls the MRI apparatus to operate in the low power mode.

7. The MRI apparatus of claim 4, wherein, when an operation of at least one of the gradient amplifier and the amplifier connected to the RF coil is not detected for a predetermined time, the control unit controls the MRI apparatus to operate in the low power mode.

8. The MRI apparatus of claim 1, wherein the control unit controls a voltage that is supplied to a motor of a patient table included in the MRI apparatus.

9. The MRI apparatus of claim 1, wherein the control unit controls an operation of at least one of a display and an audio system mounted on a gantry of the MRI apparatus.

10. The MRI apparatus of claim 1, wherein the control unit controls an operation of at least one of a gradient amplifier and an amplifier connected to a radio frequency (RF) coil, the gradient amplifier and the amplifier being included in the MRI apparatus.

11. The MRI apparatus of claim 1, wherein the control unit controls an operation of a heat exchanger included in the MRI apparatus.

12. A magnetic resonance imaging (MRI) apparatus comprising:
a monitoring unit configured to monitor status of at least one operation of the MRI apparatus;
a control unit configured to identify an associated power mode from a plurality of power modes for the MRI apparatus based on the monitored operation and control the MRI apparatus to operate in the associated power mode; and
a radio frequency (RF) coil configured to be detachably connected to at least one body part of an object,
wherein, when the RF coil is detached from the at least one body part of the object, the control unit controls the MRI apparatus to operate in a low power mode that consumes less power than the associated power mode.

13. A magnetic resonance imaging (MRI) apparatus comprising:
a monitoring unit configured to monitor status of at least one operation of the MRI apparatus; and
a control unit configured to identify an associated power mode from a plurality of power modes for the MRI apparatus based on the monitored operation and control the MRI apparatus to operate in the associated power mode; and
an input unit configured to receive a user input for selecting the associated power mode from among the plurality of power modes for the MRI apparatus.

14. The MRI apparatus of claim 13, wherein the input unit receives a user input for inputting at least one of information about an object and information about MRI scanning of the object, wherein the control unit controls the MRI apparatus to operate in a low power mode, which consumes less power than the associated power mode, while the input unit is receiving the user input.

15. A method of operating a magnetic resonance imaging (MRI) apparatus, the method comprising:
monitoring status of an MRI scanning operation by a control unit;
identifying an associated power mode from a plurality of power modes for operation of the MRI apparatus based on the monitored MRI scanning operation;
controlling the MRI apparatus to operate in the associated power mode;
changing the associated power mode to another power mode based on a change in the status of the monitored MRI scanning operation, and
controlling the MRI apparatus to operate in a low power mode that consumes less power than the associated power mode when the MRI scanning operation is completed.

16. The method of claim 15, further comprising controlling the MRI apparatus to operate in a standby mode prior to starting the MRI scanning operation.

17. The method of claim 16, further comprising controlling the MRI apparatus to operate in a quick start mode when the MRI scanning operation starts.

18. A method of operating a magnetic resonance imaging (MRI) apparatus, the method comprising:
monitoring by a control unit an operation of the MRI apparatus;
identifying an associated power mode from a plurality of power modes for the MRI apparatus based on the monitored operation;
controlling the MRI apparatus to operate in the associated power mode; and
controlling the MRI apparatus to operate in a low power mode when a radio frequency (RF) coil configured to be detachably connected to at least one body part of an object is detached from the at least one body part of the object.

19. A method of operating a magnetic resonance imaging (MRI) apparatus, the method comprising:
monitoring by a control unit a status of an operation of the MRI apparatus;
identifying an associated power mode of a plurality of power modes for the MRI apparatus based on status of the monitored operation;
controlling the MRI apparatus to operate in the associated power mode; and
receiving a user input for selecting the associated power mode from among the plurality of power modes for the MRI apparatus.
